(19) United States
(12) Patent Application Publication
(10) Pub. No.: US 2010/0068312 A1
(43) Pub. Date: Mar. 18, 2010

(54) FORMULATION FOR FEVER REDUCING TREATMENT AND A METHOD AND KIT FOR USING THEREOF

(75) Inventor: Mazal EZYON, Ashkelon (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: MICRODEL IDEA CENTER LTD., RAMAT GAN (IL)

(21) Appl. No.: 12/611,121

(22) Filed: Nov. 3, 2009

Related U.S. Application Data
(63) Continuation-in-part of application No. PCT/IL2008/000666, filed on May 14, 2008.
(60) Provisional application No. 60/939,098, filed on May 21, 2007.

(51) Int. Cl. A61K 36/185; A61K 36/13; A61P 29/00

(52) U.S. Cl. 424/725

(57) ABSTRACT

A fever reducing formulation comprising an aqueous solution of acetic acid, sweating inducing plant extracts and fragrant masking ingredients. The solution of acetic acid and the plant extract reduce fever while the fragrant masking ingredients mask the smell of the acetic acid. A kit for reducing fever comprising the above mentioned fever reducing formulation, as well as a container for mixing the formulation, an absorbing material for applying the formulation to the skin and an information and instruction sheet. A method for reducing fever comprising mixing the formulation, moistening an absorbing material with the formulation and applying the absorbing material to the skin.

### CROSS-REFERENCE TO RELATED APPLICATIONS

This application is a continuation in part of PCT/IL2008/000666 filed on May 14, 2008, and also claims benefit under 35 U.S.C. §119(e) of U.S. Provisional Patent Application No. US60/939,098 filed May 21, 2007, the content of which is incorporated by reference herein.

### FIELD OF THE INVENTION

The present invention generally relates to the field of medical treatments. More particularly, the present invention relates to a fever reducing treatment by external means.

### BACKGROUND OF THE INVENTION

Fever, as commonly defined is body temperature higher than normal. Fever occurs when the hypothalamus, which is the part of the brain that controls and regulates the temperature of the body, raises the temperature over the normal level of about 37 degrees Celsius. The hypothalamus, while serving as the body's thermostat, sets the body temperature to a higher level when an infection or another ailment is detected.

The white cells contained in the blood produce a substance called interleukin-1 when they digest microorganisms that may cause illness. Interleukin-1 impels the formation of prostaglandins that are the substances that directly trigger the hypothalamus to raise the body temperature and form fever. Thus, fever is one of the body's procedures for fighting pathogens. Consequently, there is usually no real reason to treat fever under 38 degrees Celsius.

It is also known that prior art fever reducing treatments, also acknowledged as antipyretic treatments of the Conventional Medicine, are based on oral application of medicines that belong to one of the four basic categories of medications: Aspirin, Ibuprofen, Acetaminophen, and Naproxen. These prior art antipyretic drugs additionally have a broad systemic activity therefore they are also used to relieve pains (analgesic), heal inflammations and cure swellings.

All these four basic categories of medications, which are classified as non-steroidal anti-inflammatory drugs (NSAID), reduce fever by blocking the activity of the cyclooxygenase (COX) enzyme, thus inhibiting the synthesis of prostaglandin E2 in the central nervous system, consequently blocking peripheral pain impulses. It should be emphasized that medicines that belong to these categories are effective for only minor pain, inflammations and fever.

### SUMMARY OF THE INVENTION

The present invention discloses a fever reducing formulation comprising an aqueous solution comprising acetic acid at a concentration between 1% and 5%, at least one fragrant masking ingredient, at least one emulgator, and at least one sweating inducing plant extract. The solution and the plant extract are arranged to reduce fever while the at least one masking ingredient and at least one emulgator are arranged to mask the smell of the acetic acid. The present invention further discloses a method of reducing fever comprising the steps of mixing at least one fever reducing formulation with at least one fragrant masking formulation, moistening an absorbing material with the mixed formulation, and applying the absorbing material on a part of the body. The present invention further discloses a kit for reducing fever comprising an aqueous solution comprising acetic acid at a concentration between 1% and 5%, at least one fragrant masking ingredient, at least one emulgator, at least one sweating inducing plant extract, a container for combining the aqueous solution of acetic acid, the at least one fragrant masking ingredient, the at least one emulgator and the at least one sweating inducing plant extract, as well as an absorbing material for applying the combination to the skin, and an information and instruction sheet.

In embodiments, the absorbing material comprises any combination of one or more of the following: a patch, a pad, a plaster, a towel, a cloth, a paper. In embodiments, the sweating inducing plant extract comprises an extract of at least one or any combination of one or more of the following: Acalypha spp., Piper spp., Tilia spp., Achillea spp., Thymus vulgaris, Dodonea viscose. In embodiments, the absorbing material is moistened with a fever reducing formulation and with one or more fragrant masking ingredients and a preserving substance and is enclosed in a sealed package in a ready-to-use form. Upon opening the package, the moistened absorbing material may be immediately applied to the body.

### BRIEF DESCRIPTION OF THE DRAWINGS

The subject matter regarded as the invention will become more clearly understood in light of the ensuing description of embodiments herein, given by way of example and for purposes of illustrative discussion of the present invention only, with reference to the accompanying drawings (Figures, or simply “FIGS.”), wherein:

FIG. 1 is a flowchart illustrating a method for reducing fever, according to some embodiments of the invention; and

FIG. 2 is an illustration of a kit for reducing fever, according to some embodiments of the invention.

### DETAILED DESCRIPTIONS OF SOME EMBODIMENTS OF THE INVENTION

The present invention discloses a fever reducing formulation comprising an aqueous solution of acetic acid, sweating inducing plant extracts and fragrant masking ingredients. The solution of acetic acid and the plant extract reduce fever while the fragrant masking ingredients mask the smell of the acetic acid. The formulation may be used to moisten an absorbing material which may be applied to the body. According to some embodiments of the invention, the formulation may contain DERMAPHYL Solplus, polysorbate, propylene glycol or another emulgator and colloidal silica NMT 0.1% or another form of Silica to protect the skin and fragrant masking ingredients comprising essential oils, or Johnson & Johnson 370 or other fragrants.

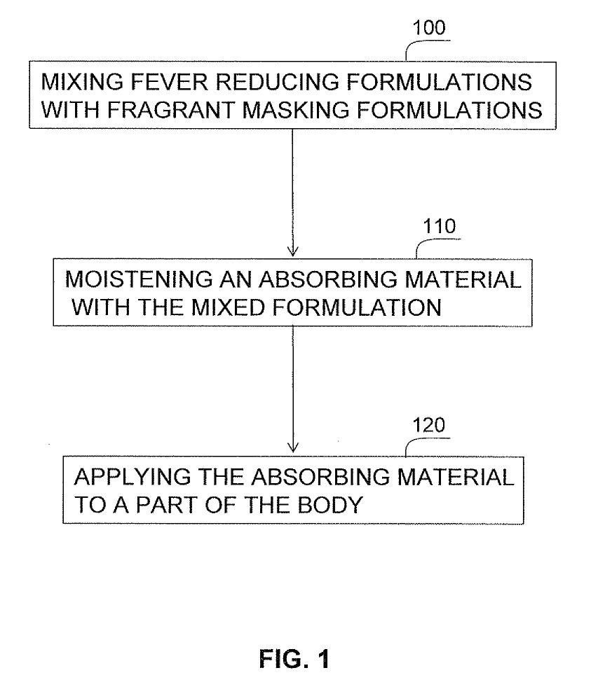

FIG. 1 is a flowchart illustrating a method for reducing fever, according to some embodiments of the invention. The method comprises the steps:
Mixing fever reducing formulations with fragrant masking formulations (step 100). Fever reducing formulations may comprise an aqueous solution of acetic acid at a concentration between 1% and 5%, sweating inducing plant extracts, such as extracts of Acalypha spp., Piper spp., Tilia spp., Achillea spp., Thymus vulgaris, Dodonea viscosa. Sweating may increase the heat dissipation from the body and thus reduce the patient's fever. Fragrant masking formulations may have a strong fragrance that mask the smell of the acetic acid solution, and may comprise pine oil, rose oil, lavender, jasmine, Limonene spp., Citronellal, spp., Coumarin or other essential oils.
Moistening an absorbing material with the mixed formulation (step 110). The absorbing material may be a patch, a pad, a plaster, a towel, or made of cloth, paper or a non-woven material.
Applying the absorbing material to a part of the body of the patient (step 120), possibly directly around the chest, back, arms or legs of the patient.

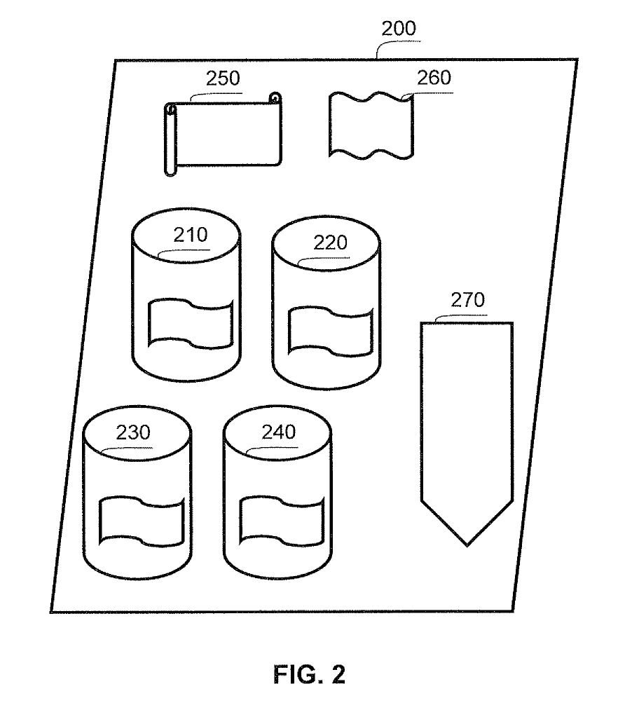

FIG. 2 is an illustration of a kit for reducing fever, according to some embodiments of the invention. The kit comprises an acetic acid vessel comprising an aqueous solution of acetic acid at a concentration between 1% and 5% 210, at least one fragrant vessel comprising at least one fragrant masking ingredient 220, at least one emulgator vessel comprising at least one emulgator, at least one extract vessel comprising at least one sweating inducing plant extract. The kit further comprises a container 270 for combining and mixing the aqueous solution of acetic acid, fragrant masking ingredients, emulgators and sweating inducing plant extracts. The kit further comprises an absorbing material 250 and an information and instruction sheet. The kit is contained in a package 200. The solution of acetic acid and plant extracts reduce fever while the fragrant masking ingredients mask the smell of the acetic acid. At least one emulgator is chosen and configures to form an emulation from the solution of acetic acid, fragrant masking ingredient and plant extract.

According to some embodiments of the invention, the absorbing material 250 may be a patch, a pad, a plaster, a towel, or made of cloth, paper or a non-woven material. The sweating inducing plant extracts may comprise extracts of Acalypha spp., Piper spp., Tilia spp., Achillea spp., Thymus vulgaris, Dodonea viscosa, or the combination of one or more thereof. The fragrant masking formulations may have a strong fragrance that mask the smell of the acetic acid solution, and may comprise pine oil, rose oil, lavender, jasmine, Limonene spp., Citronellal, spp., Coumarin, or other essential oils or such fragrant as Johnson & Johnson 370.

According to some embodiments of the invention, the absorbing material 250 may be moistened with an aqueous solution of acetic acid at a concentration between 1% and 5%, at least one fragrant masking ingredient, a preserving substance and at least one emulgator, and packed within a sealed package in a ready-to-use form. Opening the package, the moistened absorbing material may be immediately applied to the body. According to some embodiments of the invention, the emulgator may be any combination of one or more of DERMAPHYL Solplus, polysorbate, propylene glycol or other emulgators.

According to some embodiments of the invention, the fever reducing formulation may comprise colloidal silica NMT 0.1% or another form of Silica to protect the skin.

According to some embodiments of the invention, the disclosed invention avoids undesirable side effects of prior art oral application medicines, such as nausea, and hemorrhage and diarrhea in the digestive system. A single high dose of Acetaminophen, for instance, may cause stomach and kidney problems, and, as a result be fatal. This may occur since both of these organs require COX for their normal functioning. It should be stressed that these medicines are especially dangerous for small children. Acetaminophen Ibuprofen and Aspirin, for example, are not given to children under 6 month, and can be dosed at time intervals of at least 6 hours, since they may develop health problems including Reyes syndrome that causes serious damage to the brain and the liver. Additionally, chronic use of the common fever and pain relief drugs can result in liver and kidney failure. Aspirin may also trigger an asthma attack in people with asthma.

Skin plays an important role in the dissipation of heat from the body, and is thus taking part in the mechanism of fever control determined by the hypothalamus. Body temperature is increased by prevention of heat loss due to vasoconstriction (narrowing of blood vessels) and reduced sweat. According to some embodiments of the invention, the disclosed invention avoids the side effects of the oral application medicines by applying a formulation to the skin by an external mean such as a patch, pad, plaster and towel. These are particularly significant for use in children, where the antipyretic activity is a prominent objective of medicinal use of the above medicines.

Methods of treatment that act directly on the skin have the advantage of acting directly on one of the organs implicated in the production and dissipation of fever without affecting internal organs such as the liver and the stomach. One of these common folk methods is the use of a cloth soaked with low concentrated acetic acid, or vinegar, applied directly around the chest, back, arms or legs of the patient.

However, to many people, the smell of vinegar or acetic acid is considered to be unpleasant, therefore it is desirable to add, according to some embodiments of the invention, masking ingredients into the vinegar or acetic acid. These masking ingredients are substances that have a very strong fragrance that covers the original smell. These substances may be substances generally used in the cosmetic and body care industries such as pine oil, rose oil, lavender, jasmine, etc.

Breaking of fever is associated with vasodilatation (widening of blood vessels) and sweating. According to some embodiments of the invention, sweating inducing plant extracts may be added to enhance fever reduction. Plant extracts may be extracts of at least one or more of the following Acalypha spp., Piper spp., Tilia spp., Achillea spp., Thymus vulgaris, Dodonea viscosa.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention can be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Those skilled in the art will envision other possible variations, modifications, and applications that are also within the scope of the invention. Accordingly, the scope of the invention should not be limited by what has thus far been described, but by the appended claims and their legal equivalents.

What is claimed is:

1. A fever reducing formulation comprising:
an aqueous solution comprising acetic acid at a concentration between 1% and 5%,
at least one fragrant masking ingredient,
at least one emulgator,
at least one sweating inducing plant extract,
wherein said solution and said plant extract are arranged to reduce fever while said at least one masking ingredient and said at least one emulgator are arranged to mask the smell of said acetic acid.

2. The formulation of claim 1, wherein said at least one sweat inducing plant extract comprises an extract of at least one or more of the following: Acalypha spp., Piper spp., Tilia spp., Achillea spp., Thymus vulgaris, Dodonea viscosa.

3. The formulation of claim 1, wherein said at least one fragrant masking ingredient comprises any combination of at least one or more of the following: pine oil, rose oil, lavender, jasmine, Limonene spp., Citronellal, spp., Coumarin, essential oils or Johnson & Johnson 370.

4. The fever reducing formulation of claim 1, wherein said at least one emulgator comprises any combination of one or more of the emulgators DERMAPHYL Solplus, polysorbate, propylene glycol, and wherein said fever reducing formulation further may comprise a silica such as colloidal silica NMT 0.1% and a preserving substance.

5. A method of reducing fever, said method comprising:
mixing at least one fever reducing formulation with at least one sweat inducing plant extract, with at least one fragrant masking formulation, with at least one emulgator, with at least one silica,
moistening an absorbing material with said mixed formulation,
applying said absorbing material on a part of the body.

6. The method of claim 5, wherein said absorbing material is any combination of one or more of the following: a patch, a pad, a plaster, a towel, a cloth, a paper.

7. The method of claim 5, wherein said at least one fever reducing formulation comprises an aqueous solution comprising acetic acid at a concentration between 1% and 5%.

8. The method of claim 5, wherein said at least one fever reducing formulation further comprises at least one sweating inducing plant extract, wherein said at least one sweating inducing plant extract comprises an extract of at least one or more of the following: Acalypha spp., Piper spp., Tilia spp., Achillea spp., Thymus vulgaris, Dodonea viscose.

9. A kit for reducing fever, said kit comprising:
an aqueous solution comprising acetic acid at a concentration between 1% and 5%,
at least one fragrant masking ingredient,
at least one emulgator,
at least one sweating inducing plant extract,
a container for combining said aqueous solution of acetic acid, said at least one fragrant masking ingredient, said at least one emulgator and said at least one sweating inducing plant extract,
an absorbing material for applying said combination to the skin,
an information and instruction sheet,
wherein said solution of acetic acid and said at least one plant extract are arranged to reduce fever while said at least one masking ingredient and at least one emulgator are arranged to mask the smell of said acetic acid.

10. The kit of claim 9, wherein said at least one sweating inducing plant extract comprises an extract of at least one of or any combination of one or more of the following: Acalypha spp., Piper spp., Tilia spp., Achillea spp., Thymus vulgaris, Dodonea viscose.

11. The kit of claim 9, wherein said absorbing material is any combination of one or more of the following: a patch, a pad, a plaster, a towel, a cloth, a paper.

12. An absorbing material to be moistened with a fever reducing solution combined with at least one fragrant masking ingredient and with at least one emulgator, for reducing fever, wherein said absorbing material is any combination of one or more of the following: a patch, a pad, a plaster, a towel, a cloth, a paper.

13. The absorbing material of claim 12, wherein said fever reducing solution comprises an aqueous solution of acetic acid at a concentration between 1% and 5% and wherein said fever reducing solution comprises at least one sweating inducing plant extract, wherein said at least one sweating inducing plant extract comprises an extract of at least one or more of the following: Acalypha spp., Piper spp., Tilia spp., Achillea spp., Thymus vulgaris, Dodonea viscosa.

14. The absorbing material of claim 12, wherein said fragrant masking ingredient comprises any combination of at least one or more of the following: pine oil, rose oil, lavender, jasmine, Limonene spp., Citronellal, spp., Coumarin, essential oils or Johnson & Johnson 370, and said emulgator comprises a combination of at least one or more of DERMAPHYL Solplus, polysorbate, propylene glycol.

15. The absorbing material of claim 13, wherein said absorbing material moistened with a fever reducing solution and with at least one fragrant masking ingredient, and with a silica and with a preserving substance is enclosed in a sealed package in a ready-to-use form, and wherein opening the package, said moistened absorbing material may be immediately applied to the body.
